(19) United States
(12) Patent Application Publication
(10) Pub. No.: US 2025/0302454 A1
(43) Pub. Date: Oct. 2, 2025

(54) SAMPLE MANAGEMENT FOR CORE NEEDLE BIOPSY DEVICE

(71) Applicant: Devicor Medical Products, Inc., Cincinnati, OH (US)

(72) Inventors: David C. McBREEN, West Chester, OH (US); Justin REBELLINO, Cincinnati, OH (US); Andrew P. NOCK, Dayton, OH (US)

(21) Appl. No.: 19/233,075

(22) Filed: Jun. 10, 2025

Related U.S. Application Data
(63) Continuation of application No. PCT/US2023/083843, filed on Dec. 13, 2023.
(60) Provisional application No. 63/435,649, filed on Dec. 28, 2022.

(51) Int. Cl. A61B 10/02; A61B 10/00

(52) U.S. Cl. CPC A61B 10/0275; A61B 10/0096; A61B 2010/0208

(57) ABSTRACT

A core needle biopsy device, including: a needle assembly, the needle assembly including a piercer and a hollow cutter, the piercer including a sharp distal tip and a notch proximal to the distal tip, the piercer being slidably disposed within the cutter to sever a tissue sample into the notch of the piercer; a drive assembly configured to selectively move the piercer and the cutter; and a tissue sample holder including a sample chamber, a fluid source, and a nozzle, the fluid source being configured to eject a liquid or gas from the nozzle and against a portion of the biopsy device to manipulate a severed tissue sample into the sample chamber.

### PRIORITY

This application is a continuation of International Application No. PCT/US2023/083843, entitled “Sample Management for Core Needle Biopsy Device,” filed Dec. 13, 2023, which claims priority to U.S. Provisional Patent App. No. 63/435,649, entitled “Sample Management for Core Needle Biopsy Device,” filed on Dec. 28, 2022, the disclosures of which are hereby incorporated by reference herein.

### BACKGROUND

A biopsy is the removal of a tissue sample from a patient to enable examination of the tissue for signs of cancer or other disorders. Tissue samples may be obtained in a variety of ways using various medical procedures involving a variety of the sample collection devices. For example, biopsies may be open procedures (surgically removing tissue after creating an incision) or percutaneous procedures (e.g. by fine needle aspiration, core needle biopsy, or vacuum assisted biopsy). After the tissue sample is collected, the tissue sample is typically analyzed at a lab (e.g. a pathology lab, biomedical lab, etc.) that is set up to perform the appropriate tests (such as histological analysis).

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device.

One technique for collecting a breast biopsy is to use a core needle biopsy device. One such device is the MAX-CORE disposable core biopsy instrument manufactured by Bard Biopsy Systems. Core needle biopsy devices frequently use a sharp, solid piercer equipped with a lateral tissue receiving notch positioned adjacent to the distal end of the piercer. When tissue is received within the notch, an elongate hollow cutting sheath is translated over the notch to sever a tissue sample. The severed tissue sample is then stored within the notch until both the piercer and the cutting sheath are removed from the patient. Thus, in core-needle biopsy devices, only one tissue sample can be collected per insertion of the piercer and cutting sheath.

In contrast to core needle breast biopsy procedures, vacuum-assisted breast biopsy devices permit the probe to remove multiple samples without requiring the probe be removed from the breast after every sample is collected. For instance, in a vacuum assisted breast biopsy device, a hollow needle is used to penetrate tissue. The hollow needle includes a lateral aperture adjacent to a sharp distal tip. A hollow cutter is disposed within the hollow needle and is moved axially relative to the lateral aperture of the needle to sever tissue samples. Once a tissue sample is severed by the hollow cutter, the tissue sample is transported axially though the cutter and collected in a tissue collection feature.

Examples of vacuum assisted biopsy devices and biopsy system components are disclosed in U.S. Pat. No. 5,526,822, entitled “Method and Apparatus for Automated Biopsy and Collection of Soft Tissue,” issued Jun. 18, 1996; U.S. Pat. No. 6,086,544, entitled “Control Apparatus for an Automated Surgical Biopsy Device,” issued Jul. 11, 2000; U.S. Pat. No. 6,162,187, entitled “Fluid Collection Apparatus for a Surgical Device,” issued Dec. 19, 2000; U.S. Pat. No. 6,432,065, entitled “Method for Using a Surgical Biopsy System with Remote Control for Selecting an Operational Mode,” issued Aug. 13, 2002; U.S. Pat. No. 6,752,768, entitled “Surgical Biopsy System with Remote Control for Selecting an Operational Mode,” issued Jun. 22, 2004; U.S. Pat. No. 7,442,171, entitled “Remote Thumbwheel for a Surgical Biopsy Device,” issued Oct. 8, 2008; U.S. Pat. No. 7,854,706, entitled “Clutch and Valving System for Tetherless Biopsy Device,” issued Dec. 1, 2010; U.S. Pat. No. 7,914,464, entitled “Surgical Biopsy System with Remote Control for Selecting an Operational Mode,” issued Mar. 29, 2011; U.S. Pat. No. 7,938,786, entitled “Vacuum Timing Algorithm for Biopsy Device,” issued May 10, 2011; U.S. Pat. No. 8,083,687, entitled “Tissue Biopsy Device with Rotatably Linked Thumbwheel and Tissue Sample Holder,” issued Dec. 21, 2011; U.S. Pat. No. 8,118,755, entitled “Biopsy Sample Storage,” issued Feb. 1, 2012; U.S. Pat. No. 8,206,316, entitled “Tetherless Biopsy Device with Reusable Portion,” issued on Jun. 26, 2012; U.S. Pat. No. 8,702,623, entitled “Biopsy Device with Discrete Tissue Chambers,” issued on Apr. 22, 2014; U.S. Pat. No. 8,858,465, entitled “Biopsy Device with Motorized Needle Firing,” issued Oct. 14, 2014; and U.S. Pat. No. 9,326,755, entitled “Biopsy Device Tissue Sample Holder with Bulk Chamber and Pathology Chamber,” issued May 3, 2016. The disclosure of each of the above-cited U.S. Patents is incorporated by reference herein.

Additional examples of vacuum assisted biopsy devices and biopsy system components are disclosed in U.S. Pub. No. 2006/0074345, entitled “Biopsy Apparatus and Method,” published Apr. 6, 2006 and now abandoned; U.S. Pub. No. 2009/0131821, entitled “Graphical User Interface for Biopsy System Control Module,” published May 21, 2009, now abandoned; U.S. Pub. No. 2010/0152610, entitled “Hand Actuated Tetherless Biopsy Device with Pistol Grip,” published Jun. 17, 2010, now abandoned; U.S. Pub. No. 2010/0160819, entitled “Biopsy Device with Central Thumbwheel,” published Jun. 24, 2010, now abandoned; and U.S. Pub. No. 2013/0324882, entitled “Control for Biopsy Device,” published Dec. 5, 2013. The disclosure of each of the above-cited U.S. Patent Application Publications is incorporated by reference herein.

Examples of core needle biopsy devices are disclosed in U.S. Pat. No. 5,560,373, entitled “Needle Core Biopsy Instrument with Durable or Disposable Cannula Assembly,” issued on Oct. 1, 1996; U.S. Pat. No. 5,817,033, entitled “Needle Core Biopsy Device,” issued on Oct. 6, 1998; U.S. Pat. No. 5,971,939, entitled “Needle Core Biopsy Device,” issued on Oct. 26, 1999; and U.S. Pat. No. 5,511,556, entitled “Needle Core Biopsy Instrument,” issued on Apr. 30, 1996. The disclosure of each of the above-cited U.S. Patents is incorporated by reference herein.

In some examples, it may be desirable to combine features from a core needle biopsy device and a vacuum assisted biopsy device to obtain the advantage of both devices and also reduce the overall disadvantages. For instance, core needle biopsy devices may be advantageous for their simplicity, light weight, and maneuverability. Furthermore, core needle biopsy devices generally include smaller sized needles, which can be desirable to increase patient comfort and recovery times. Meanwhile, vacuum assisted biopsy devices may be advantageous for their ability to collect multiple samples in a single insertion. Thus, a simple and light weight biopsy device capable of collecting multiple samples with a single insertion may be desirable.

One challenge in use of biopsy devices may include management of tissue samples once they are collected using the biopsy device. In some cases where a core needle biopsy device is used, challenges can arise due to the unique needle and cutter configuration. For instance, the cutter may be on the exterior of an inner piercer, stylet, or needle. A notch in the inner piercer can then be used to transport a severed tissue sample through the cutter. While the use of the notch can improve sample acquisition in some scenarios, collection of the severed tissue sample from the notch can be challenging due to the size and/or shape of the notch as well as the characteristics of the severed tissue sample (e.g., “sticky” or “clingy”). Thus, certain tissue sample collection features may be desirable for integration into a biopsy device that combines features of core needle biopsy devices and vacuum assisted biopsy devices.

While several systems and methods have been made and used for obtaining a biopsy sample, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of a version of a core needle biopsy device.

FIG. 2 depicts a perspective exploded view of a needle assembly of the core needle biopsy device of FIG. 1.

FIG. 3 depicts a schematic view of and the needle assembly of FIG. 2 together with a drive assembly and a tissue handler.

FIG. 4 depicts a schematic view of the tissue handler of FIG. 3, the tissue handler including an extraction mechanism.

FIG. 5 depicts perspective view of another extraction mechanism, the extraction mechanism being incorporated into the drive assembly of FIG. 3.

FIG. 6 depicts an exploded perspective view of the extraction mechanism of FIG. 5.

FIG. 7 depicts a detailed perspective view of a retraction mechanism for use with an actuator of the extraction mechanism of FIG. 5.

FIG. 8A depicts a cross-sectional series view of the extraction mechanism of FIG. 5, the actuator of the extraction mechanism being in a cocked configuration.

FIG. 8B depicts another cross-sectional series view of the extraction mechanism of FIG. 5, the actuator of the extraction mechanism in a fired configuration.

FIG. 9 depicts a perspective view of yet another extraction mechanism for incorporation into the biopsy device of FIG. 1.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Biopsy devices may be used to collect tissue samples in a variety of ways. For example, in some instances tissue samples are collected into a single tissue basket such that all tissue samples collected during a given biopsy procedure are deposited into the single tissue sample basket. In some other instances, tissue samples are collected into a tissue sample holder having separate compartments for each collected tissue sample. Such a multi-compartment tissue sample holder may additionally include trays or strips that individually hold each tissue sample separately from the other tissue samples. Such trays or strips may be removable or otherwise separable from the tissue sample holder at the conclusion of a biopsy procedure.

Regardless of the structure in which the tissue samples are stored, tissue samples may be collected using biopsy devices under the guidance of various imaging modalities such as ultrasound image guidance, stereotactic (X-ray) guidance, MRI guidance, Positron Emission Mammography (“PEM” guidance), Breast-Specific Gamma Imaging (“BSGI”) guidance, or otherwise. Each procedure has its own methodology based on the form of imaging guidance used.

Vacuum assisted biopsy devices and core needle biopsy devices both may have various advantages over the other, depending on context. For instance, one advantage of vacuum assisted biopsy devices is that vacuum assistance permits removal of multiple tissue samples using a single insertion. However, while core needle biopsy devices lack this feature, use of core needle biopsy devices may still be desirable in some scenarios. For instance, core needle biopsy devices may be generally capable of having smaller needles relative to vacuum assisted biopsy devices, thereby reducing patient anxiety and increasing the capacity of the needle to penetrate a lesion. Therefore, in some instances it may be desirable to incorporate the feature of multiple sample removal of a vacuum assisted biopsy device into a core needle biopsy device to achieve benefits present in both styles of biopsy device.

A desirable feature of the device described herein, which is a core needle biopsy device, is that the device allows for single insertion with multiple samples being obtained whilst using a core needle type device. To facilitate this functionality, the biopsy device further includes a tissue sample holder having one or more features to facilitate collection of a severed tissue sample from a notch, dugout, aperture, and/or other sample collection feature.

### I. Example Core Needle Biopsy Device With Multi-Sample Collection

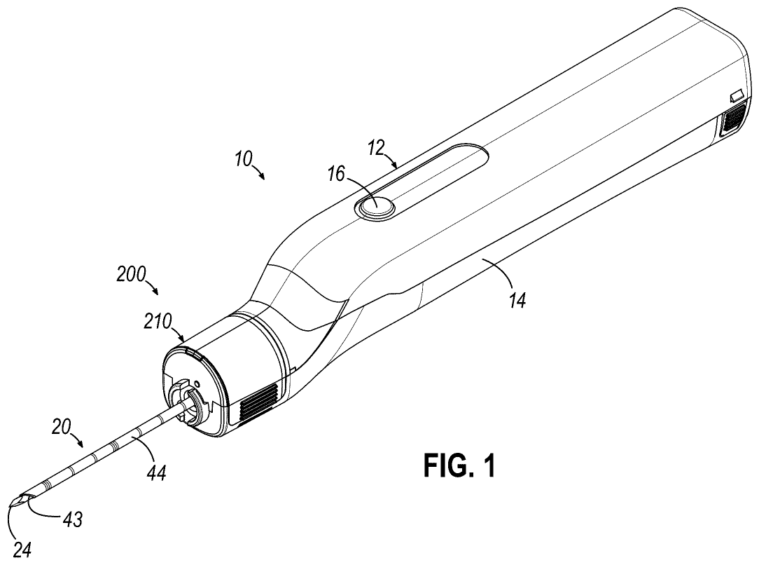

FIGS. 1 shows a version of a core needle biopsy device (10) for use in a breast biopsy procedure. Core needle biopsy device (10) of the present version comprises a body (12) and a needle assembly (20) extending distally from body (12). Body (12) includes an outer housing (14) and an actuation member (16) disposed on outer housing (14). As will be described in greater detail below, outer housing (14) encloses various components of biopsy device (10), which are used to drive needle assembly (20) through a cutting cycle and a tissue acquisition cycle. To this end, outer housing (14) of the present version is sized and shaped for grasping by an operator using a single hand. Although not shown, it should be understood that in some versions outer housing (14) may comprise multiple parts such that each part interconnects to form outer housing (14).

### A. Example Needle Assembly

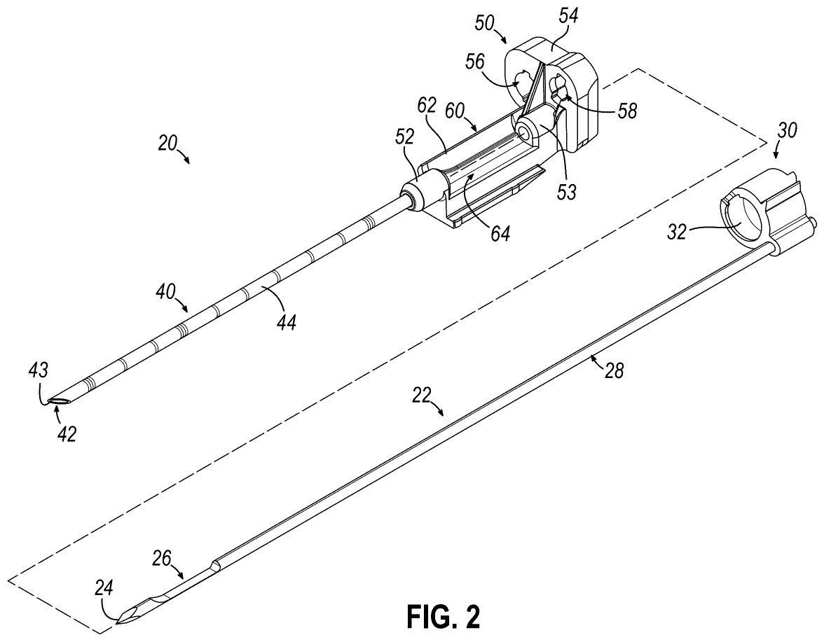

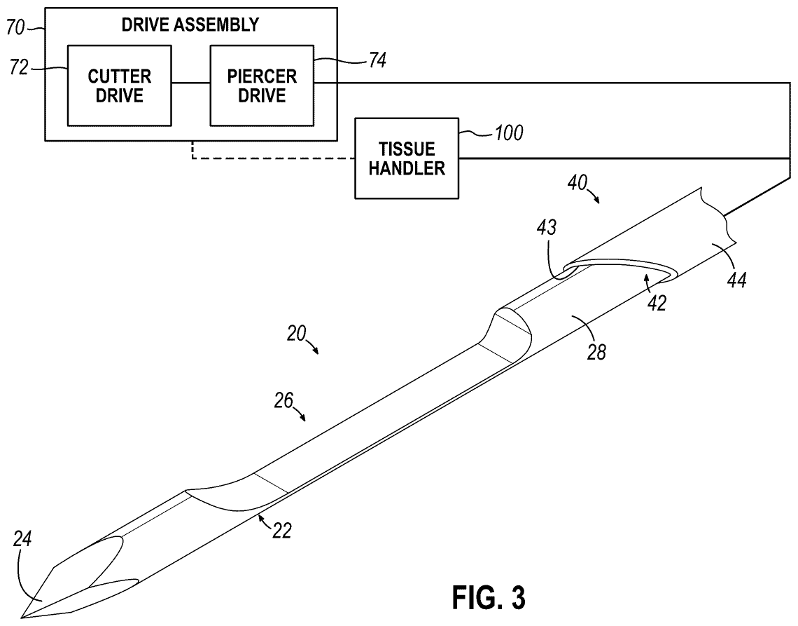

FIGS. 2 and 3 show needle assembly (20) in greater detail. As can be seen in FIG. 2, needle assembly (20) comprises an elongate piercer (22) and an elongate cutter (40). As will be described in greater detail below, piercer (22) is generally movable relative to cutter (40) to pierce tissue and collect tissue samples, while cutter is generally movable relative to piercer (22) to sever tissue samples. Piercer (22) comprises a generally cylindrical rod (28) (also referred to as a shaft) having a sharp distal tip (24) and a notch (26) disposed proximate distal tip (24). As will be described in greater detail below, distal tip (24) is generally configured to penetrate tissue of a patient. As will also be described in greater detail below, notch (26) is generally configured to receive tissue therein such that a tissue sample may be collected within notch (26) after the tissue sample is severed by cutter (40).

An end portion (30) is disposed on the proximal end of piercer (22). End portion (30) of the present version is overmolded, or otherwise fixedly secured to, the proximal end of piercer (22) and is generally configured to enhance the manipulability of piercer (22). In particular, end portion (30) comprises a receiving feature (32) in the form of a cylindrical recess. Receiving feature (32) is configured to receive a portion of a piercer drive assembly (130). Thus, receiving feature (32) in the present version may be configured as a carriage or other engagement feature configured to facilitate drive of piercer (22). As will be described in greater detail below, this permits piercer drive assembly (130) to drive movement of piercer (22) through a predetermined sequence of movement.

Cutter (40) comprises a generally hollow cylindrical tube that is configured to receive piercer (22) therein. Cutter (40) comprises an open distal end (42), a cannula portion (44) and an end portion (50). Open distal end (42) is configured to permit at least a portion of piercer (22) to protrude from cutter (40) when piercer (22) is moved relative to cutter (40). In some versions, such as the version shown, open distal end (42) may also be oriented at an angle relative to the longitudinal axis of cutter (40). In other versions, open distal end (42) may alternatively be perpendicular relative to the longitudinal axis of cutter (40). As will be described in greater detail below, this configuration permits needle assembly (20) to move through the cutting cycle and the tissue acquisition cycle by permitting notch (26) of piercer (22) to move relative to distal end (42) of cutter (40).

Open distal end (42) of the present version includes a tapered edge (43). Tapered edge (43) is generally configured to slice through tissue to separate tissue samples when cutter (40) is moved relative to notch (26) of piercer (22). Thus, it should be understood that tapered edge (43) is generally configured to act a blade. Although the present version is described and shown as using a tapered configuration, it should be understood that in other versions various alternative configurations can be used. For instance, in some versions tapered edge (43) includes a plurality of serrations in addition or in alternative to the taper shown. In still other versions, tapered edge (43) can include any other additional or alternative cutting surface as will be apparent to those of ordinary skill in the art in view of the teachings herein.

Cannula portion (44) of cutter (40) extends proximally from distal end (42) through end portion (50) such that piercer (22) can be received with the proximal end of cutter (40). Unlike end portion (30) of piercer (22), end portion (50) of cutter (40) is generally elongate such that end portion (50) can accommodate additional features that will be described in greater detail below. In the present version, the distal extension of end portion (50) can be relative to outer housing (14) to permit a portion of end portion (50) to be accessible to an operator for tissue sample collection purposes. Various suitable tissue collection mechanisms associated with end portion (50) will be described in greater detail below.

End portion (50) of cutter (40) comprises a cutter collar (52), a piercer collar (53), drive feature (54) and a tissue collection feature (60) (also referred to as a ledge, primary ledge, wiper catch, or wiper engagement feature) disposed between the cutter collar (52) and the drive feature (54). Cutter collar (52) is generally configured to receive the proximal end of cutter (40) to fixedly secure cutter (40) to end portion (50). Additionally, cutter collar (52) is configured to promote access of piercer (22) to cutter (40). In this configuration, piercer (22) may be slidably disposed within end portion (50) and extend distally through cutter (40). As will be described in greater detail below, this may permit piercer (22) to slide within cutter (40) and end portion (50) proximally to retract notch (26) from the proximal end of cutter (40) and thereby expose notch (26) within tissue collection feature (60).

Piercer collar (53) extends distally from a portion of drive feature (54) and is generally configured to slidably receive a portion of piercer (22). In particular, piercer collar (53) is positioned along a common axis with cutter (40) and cutter collar (52) to align piercer (22) with cutter (40) along the same common axis. Thus, cutter (40), cutter collar (52), and piercer collar (53) may be configured to act cooperatively to maintain piercer (22) along a common axis. As will be described in greater detail below, this feature may be desirable during use to remove a tissue sample from notch (26) when notch is disposed within tissue collection feature (60).

Drive feature (54) of end portion (50) is generally configured to engage features of a cutter drive assembly (120) for manipulation of cutter (40) via drive end portion (50) through a predetermined sequence of movement. Although various suitable configurations may be used, in the present version, drive feature (54) includes a drive opening (56) and a release opening (58). Each opening (56, 58) may be configured to engage corresponding components of cuter drive assembly (120) to permit manipulation of cutter (40) via end portion (50).

Tissue collection feature (60) is disposed distally relative to drive feature (54). Tissue collection feature (60) generally defines an elongate notch that is open, or otherwise exposed, relative to cannula portion (44) of cutter (40). In the present version, the proximal end of cutter (40) is disposed distally of tissue collection feature (60) to expose the interior of cutter (40) relative to tissue collection feature (60). However, it should be understood that in other versions, cannula portion (44) may include a cutout, opening, lateral aperture, or other feature that may be adjacent to, or otherwise define, tissue collection feature (60). Regardless, it should be understood that tissue collection feature (60) is in communication with the hollow interior, or a lumen, defined by cannula portion (44). As will be described in greater detail below, this relationship between tissue collection feature (60) and cannula portion (44) permits an operator to remove tissue samples from cutter (40) as they are collected by piercer (22).

Tissue collection feature (60) includes an engagement ledge (62) (also referred to as a protrusion, primary ledge, stopping member, or tensioner) and a recessed portion (64). Engagement ledge (62) extends upwardly relative to cutter collar (52) and piercer collar (53), defining a curved or wave-shaped profile. Engagement ledge (62) further extends longitudinally along the length of tissue collection feature (60) or from cutter collar (52) to piercer collar (53). As will be described in greater detail below, engagement ledge (62) is generally configured to engage one or more portions of a tissue sample holder (17) to facilitate transfer of a tissue sample from notch (26) of piercer (22) into tissue sample holder (17).

Recessed portion (64) is positioned adjacent or proximate to engagement ledge (62) and extends from cutter collar (52) to piercer collar (53). Recessed portion (64) is generally configured to receive piercer (22) therein to provide access to notch (26) of piercer (22). As such, recessed portion (64) defines a curved shape that may be complementary to the shape of piercer (22).

FIG. 3 shows piercer (22) disposed within cutter (40) and being in communication with a schematic view of a drive assembly (70) and tissue handler (100). Drive assembly (70) may include a cutter drive (72) configured to translate cutter (40) and a piercer drive (74) configured to translate piercer (22). As can be seen, cutter (40) is generally configured to receive piercer (22) such that piercer (22) is coaxial with cutter (40). In addition, piercer (22) is generally movable relative to open distal end (42) of cutter (40). It should be understood that in some circumstances piercer (22) moves relative to cutter (40) through operation of piercer drive (74), while cutter (40) remains stationary. In other circumstances, cutter (40) moves relative to piercer (22) through operation of cutter drive (72), while piercer (22) remains stationary. In either case, it should be understood that piercer (22) and cutter (40) are generally configured such that notch (26) of piercer (22) moves into and out of cutter (40) such that notch (26) can be disposed distally or proximally relative to open distal end (42) of cutter (40). As will be described in greater detail below, this configuration permits piercer (22) and cutter (40) to operate cooperatively to pierce tissue, cut a tissue sample, and retract the tissue sample for collection by an operator via tissue collection feature (60). In some versions drive assembly (70) may be configured in accordance with one or more of the teachings of US Pub. No. 2002/0249075, published on Aug. 11, 2022, the disclosure of which is incorporated by reference herein.

### B. Example of Fluid-Based Tissue Handler

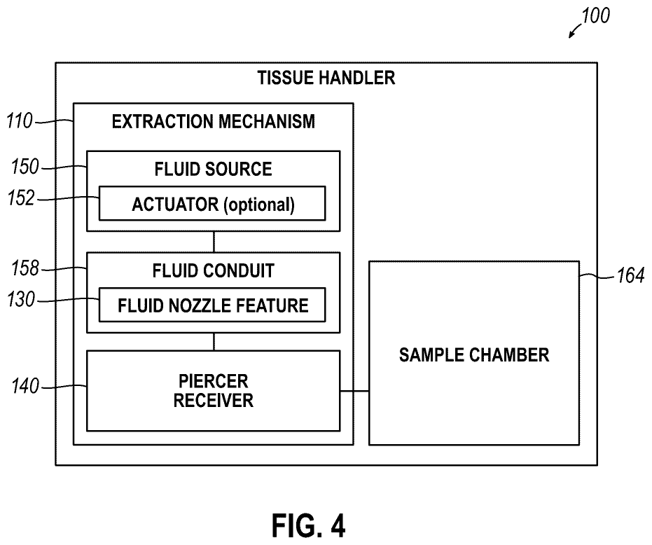

FIG. 4 shows a tissue handler (100) for extracting tissue from needle assembly (20). Tissue handler (100) may include an extraction mechanism (110) and a sample chamber (164). Extraction mechanism (110) may be configured to dislodge or otherwise remove tissue disposed within notch (26) of piercer (22). As will be described in greater detail below, extraction mechanism (110) may generally be configured to emit a fluid toward notch (26) of piercer (22), which may move a tissue sample from notch into a portion of sample chamber (164).

Extraction mechanism (110) may include a fluid source (150), a fluid conduit (158), and a piercer receiver (140). Fluid source (150) may be configured to contain or hold fluid used remove or relocate a tissue sample. Fluid source (150) may be capable of storing either a liquid or gas and may be configured as single-use or reusable and refillable. Fluid source may be located entirely or partially inside or outside of a biopsy device (10). In some versions, fluid source (150) may be detachable from biopsy device (10) such that fluid source (150) may be omitted from biopsy device (10) when not in use.

Optionally, an actuator (152) may be positioned inside or proximate fluid source (150). Actuator (152) may be generally configured to facilitate the transport of fluid from fluid source (150) and into a nearby area. In some versions, actuator (152) may include a plunger or piston similar to structures in a syringe. In other versions, actuator (152) may include a motorized pump or the like. Alternatively, fluid source (150) may omit actuator (152) and instead rely on fluid pressure within fluid source (150) for the communication of fluid.

Fluid source (150) may contain any fluid capable of being stored inside fluid source (150). Fluid may be a liquid or a gas or any combination such that fluid is capable of being expelled from fluid source (150). Some examples of fluid may be ambient air, oxygen, nitrogen, water, or saline. When in liquid form, fluid may have a viscosity so that fluid will be prevented from seeping out of fluid source (150). Seeping may be prevented simply due to a high fluid viscosity such that fluid is required to be forced out of fluid source (150). Seeping may also be prevented due to a high fluid surface tension such that fluid is incapable of exiting an orifice of fluid source (150) while simultaneously allowing an alternative medium to enter fluid source (150). In some versions, fluid source (150) can include a one-way check valve. Such a one-way check valve may permit fluid to enter the flow path fluid source (150), yet remain held in place under a partial vacuum until fluid source (150) is pressurized.

Fluid source (150) may be in fluid communication with fluid conduit (158) either directly or via an intermediate conduit or tube (not shown). Fluid conduit (158) is generally configured to permit the flow of fluid from fluid source (150) and into fluid conduit (158) to expel fluid from fluid source (150). Fluid may also be capable of flowing from fluid conduit (158) and into fluid source (150) to fill fluid source (150) with fluid. Fluid conduit (158) may be positioned adjacent to an orifice of fluid source (150). Fluid conduit (158) may be configured to reduce head loss such as by forming a straight tube or may be configured to accommodate slight misalignments or movements across fluid conduit (158) such as by including a flexible sidewall. Fluid conduit (158) may include a rigid sidewall and/or a consistent diameter such that fluid conduit (158) can maintain a consistent pressure and flow of fluid throughout. Fluid conduit (158) may include a sidewall capable of maintaining a vacuum without collapsing such as when refilling fluid source (150) by applying a vacuum at fluid source (150). Fluid conduit (158) may be incorporated into other portions of tissue handler (100) or drive assembly (70) such as in cutter drive (72), piercer drive (74), fluid source (150), or sample chamber (164), discussed later.

Fluid conduit (158) may be in fluid communication with a fluid nozzle feature (130). Fluid nozzle feature (130) may be removably coupled or integral with fluid conduit (158). Fluid nozzle feature (130) may be capable of spraying or propelling fluid through a spout, opening, orifice, or jet to thereby direct fluid in a targeted manner towards a tissue sample. Fluid nozzle feature (130) may be capable of spraying fluid in a more direct solid stream or a fanned manner in the general direction of a piercer receiver (140).

Piercer receiver (140) may be configured to position and/or expose a portion of a piercer (22) relative to fluid conduit (158) and/or fluid nozzle feature (130) to facilitate the communication of fluid from fluid conduit (158) and/or fluid nozzle feature (130) to a portion of piercer (22). Piercer receiver (140) may include one or more features configured to promote or allow the removal of tissue from portion of piercer (22). Such features may include a shelve, ledge, curved surface, opening, orifice, or channel which may direct the flow of fluid from fluid nozzle feature (130) towards piercer (22). In some versions, piercer receiver (140) may be incorporated into biopsy device (10) as one or more portions of tissue collection feature (60) described above. In still other versions, piercer receiver (140) may be omitted and nozzle feature (130) may be positioned proximate to structures such as tissue collection feature (60).

Sample chamber (164) may be positioned proximate to piercer receiver (140) and may be configured to capture and contain one or more tissue sample that have been removed from a portion of the piercer (22) by fluid ejected from fluid nozzle feature (130). In some versions, sample chamber (164) may be a fluidly sealed container such that portions of tissue sample may remain captured in sample chamber (164). Sample chamber (164) may also include a vent or a valve configured to relieve sample chamber (164) of any pressure buildup once fluid nozzle feature (130) ejects fluid. The inside of sample chamber (164) may be accessible to remove or otherwise collect tissue samples. Accessing inside sample chamber (164) may be conducted by removing a portion of sample chamber (164) such as a cap or a canister from tissue handler (100) or may be conducted by rotating or sliding a portion of sample chamber (164) to gain access to the inside. Sample chamber (164) may be configured to discretely collect each tissue sample and to hold each tissue sample separate from any other tissue sample collected.

Tissue handler (100) may be partially operable with any portion of biopsy device (10) such that operation of one component or system may drive a portion of tissue handler (100).

As an example, actuator (152) may be either released (also referred to as fired) or cocked by cutter drive (72) and/or piercer drive (74). Such integration with cutter drive (72) and/or piercer drive (74) may be desirable to coordinate movements of needle assembly (20) with operation of actuator (152). As a specific example, actuator (152) may be driven away from fluid source (150) by piercer drive (74). Once actuator (152) has reached a suitable position, actuator (152) may be released (also referred to as fired) by piercer drive (74), such that actuator (152) is driven towards fluid source (150) to thus dislodge tissue from notch (26) of piercer (22). In other versions, actuator (152) may be controlled by separate actuation mechanisms such as motor driven mechanisms, linear drives, lead screws, manually driven levers, gears, wheels, and/or plungers, and/or etc.

### C. Example of Tissue Handler With Drive Assembly Integration

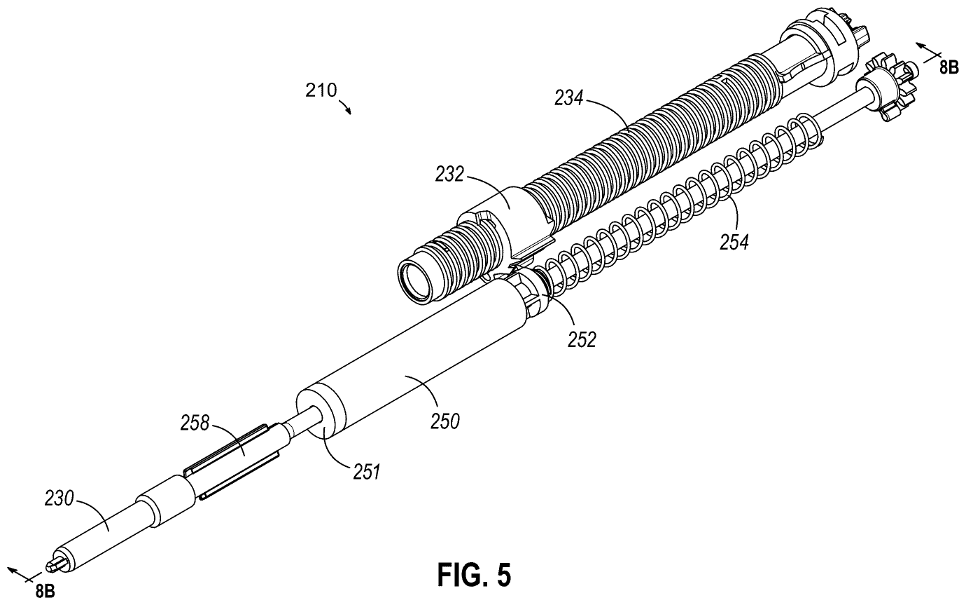

FIGS. 5-7 show an alternative extraction mechanism (210) that may be used in tissue handler (100) described above in lieu of extraction mechanism (110). As shown in FIG. 5, and will be described in greater detail below, extraction mechanism (210) of the present version is integrated into a portion of drive assembly (70) to permit extraction of tissue from needle assembly (20) using pneumatic pressure or other fluid media in coordination with drive of needle assembly (20) provided by drive assembly (70). Similar to extraction mechanism (110) described above, extraction mechanism (210) of the present version may include a fluid source (250), a fluid conduit (258), and a nozzle (230). In some versions, fluid source (250), fluid conduit (258), and nozzle (230) may be configured substantially similarly to fluid source (150), fluid conduit (158), and/or fluid nozzle feature (130) described above unless otherwise described herein.

Fluid source (250) may be in the form of a cylinder or tube capable of containing a fluid. Fluid source (250) may include an opening at a distal end for a fluid source connector (251) to fluidly connect fluid source (250) to fluid conduit (258). Fluid source (250) may be capable of capturing a volume of fluid that is capable of dislodging tissue from a piercer (22). Fluid source (250) may include a rigid sidewall configured to withstand fluid pressures without substantially deforming or expanding. Fluid source (250) may include or have attached fluid source connector (251). Fluid source connector (251) may be attached at a distal end of fluid source (250). Fluid source connector (251) may be fluidly sealed at a connector-fluid source junction and at a connector-conduit junction to thereby reduce leaking of fluid within fluid source (250).

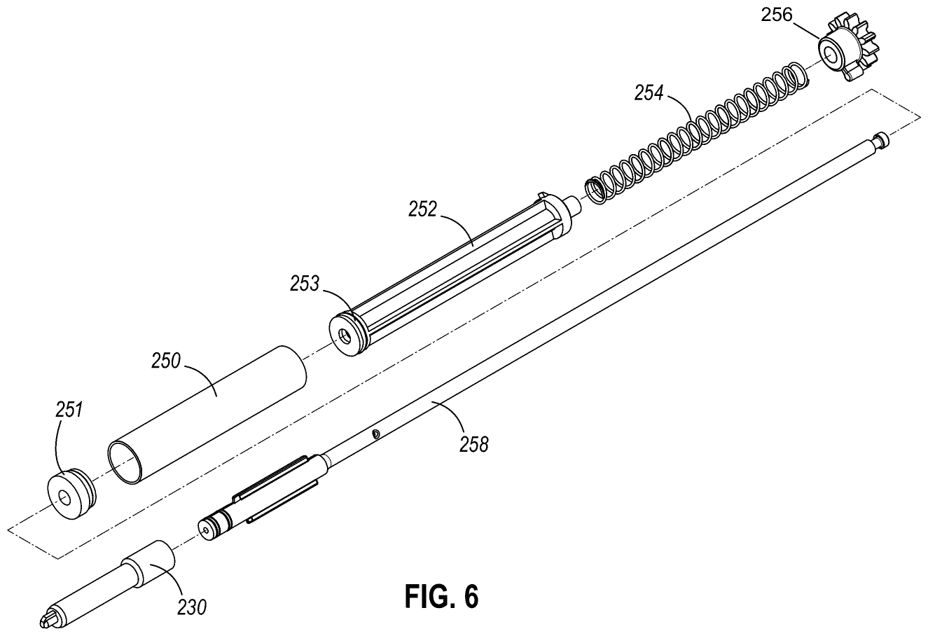

Actuator (252) is configured for receipt within the interior of fluid source (250). In particular, actuator (252) may be in the form of a piston which translates within fluid source (250). As shown in FIG. 6, actuator (252) may optionally include one or more longitudinally extending ribs. Such ribs may be configured to promote rigidity without adding substantial weight and to maintain an alignment within fluid source (250) during translation of actuator (252). As will be described in greater detail below, actuator (252) is configured to translate within fluid source (250) to displace fluid from fluid source (250) thorough fluid conduit (258) towards nozzle (230). To facilitate such displacement of fluid, actuator (252) may include a seal (253), such as a gasket or o-ring, at a distal end to sealingly engage the interior of fluid source (250).

Fluid conduit (258) may extend axially through fluid source (250) and actuator (252). In particular, fluid conduit (258) may define a generally elongate cylindrical structure. In some versions, this elongate cylindrical structure may act as a guide for actuator (252), actuator spring (254), and/or etc. For instance, actuator (252) of the present version is configured to slidably engage fluid conduit (258) to permit translation of actuator (252) relative to fluid conduit (258) and fluid source (250). Thus, fluid conduit (258) extends the length of extraction mechanism (210) in the present version, but may be less than the length of extraction mechanism (210) in other versions. As will be describe in greater detail below, fluid conduit (258) may define a lumen (253) extending through a portion thereof. Thus, fluid conduit (258) may be of a sufficiently rigid material to support lumen (253) when under pressure.

In the present version, fluid conduit (258) is optionally incorporated into certain aspects of drive assembly (70). For instance, fluid conduit (258) of the present version is incorporated into a rod-like structure that may be configured to control one or more functions of drive assembly (70) such as firing of cutter (40) and/or piercer (22) via rotation of the rod-like structure. In some versions, the rod-like structure of fluid conduit (258) may be configured in accordance with one or more teachings of US Pub. No. 2022/0249075, published Aug. 11, 2022, the disclosure of which is incorporated by reference herein. In still other versions, fluid conduit (258) may be configured entirely separately from drive assembly (70) as will be understood by those of ordinary skill in the art in view of the teachings herein.

Lumen (253) may be defined by fluid conduit (258) to span from an opening in the sidewall of fluid conduit (258) to a distal opening in fluid conduit (258). Lumen (253) may further extend along a longitudinal axis defined by fluid conduit (258). In the present version, lumen (253) only extends for a portion of the total length of fluid conduit (258). In particular, lumen (253) of the present version extends only through a distal portion of fluid conduit (258). Thus, a proximal portion of fluid conduit (258) may optionally be solid and omit structures such as lumen (253). Such a solid proximal configuration may be desirable to provide support or enhanced rigidity for actuator (252) and/or actuator spring (254). Fluid conduit (258) may include a distal end having a larger diameter than a proximal end. The larger diameter may be capable of preventing fluid source (250) from advancing distally once actuator (252) advances into fluid source (250).

Fluid conduit (258) may be fluidly coupled with nozzle (230) at a distal end of fluid conduit (258). Such a fluid coupling may be configured to permit fluid travelling through lumen (253) to reach and be expelled from nozzle (230). As described in above, in some versions, fluid conduit (258) may be rotatable to facilitate certain functions of drive assembly (70). Yet, as will be described in greater detail below, it may be desirable for nozzle (230) to remain in a fixed position. Thus, in the present version, fluid conduit (258) may be coupled to nozzle (230) with a coupling configured to permit relative rotation between fluid conduit (258) and nozzle (230), while also holding fluid conduit (258) and nozzle (230) in a fixed axial position. Such a coupling between fluid conduit (258) and nozzle (230) may also include one or more seals to fluidly seal the coupling between fluid conduit (258) and nozzle (230).

In some versions, a release gear (256) (also referred to as a drive gear) is disposed at a proximal end of fluid conduit (258). In such versions, release gear (256) is configured to rotate fluid conduit (258) using input from one or more drive mechanisms such as a motor. In some versions, rotation of fluid conduit (258) via release gear (256) may be used to facilitate one or more functions of drive assembly (70), as described above. In addition, or in the alternative, rotation of fluid conduit (258) via release gear (256) may be used to facilitate one or more aspects of actuation of actuator (252), as will be described in greater detail below.

Nozzle (230) may be secured to a portion of biopsy device (10) such that nozzle (230) is rotationally and/or longitudinally fixed relative to a sample chamber, piercer (22), and/or cutter (40). Rotationally fixing nozzle (230) allows an opening of nozzle (230) to be directed at tissue to allow for the expulsion of tissue into sample chamber without the need for operator to adjust the direction. Rotationally fixing nozzle (230) may be performed through use of an adhesive, hardware, or by integrally incorporating it into sample chamber. Nozzle (230) may be fluidly sealed to fluid conduit (258) through use of an o-ring seal, gasket, or a taper connection such that fluid conduit (258) may rotate relative to nozzle (230). Sample chamber and nozzle (230) may be removably coupled from a remaining portion of tissue handler such that any portion to come into contact with tissue may be replaced from other portions of tissue handler.

Extraction mechanism (210) further includes an actuator spring (254) arranged coaxially with fluid source (250), actuator (252), and fluid conduit (258). Actuator spring (254) may be configured to exert a force on a proximal end of actuator (252). As will be described in greater detail below, actuator spring (254) is configured to push actuator (252) into fluid source (250) to thereby expel fluid from fluid source (250). Thus, although not shown, it should be understood that a proximal end of actuator spring (254) may be axially fixed relative to actuator (252). In some versions, such an axial fixation may be facilitated by release gear (256). In still other versions, such an axial fixation may be facilitated by a portion of a housing such as housing (14) of biopsy device (10). Although actuator spring (254) of the present version is shown as a coil spring, in other versions, actuator spring (254) may be in any other form of spring or linear device capable of exerting a directional force on actuator (252).

As described above, extraction mechanism (210) of the present version is configured to be incorporated into a portion of drive assembly (70) of biopsy device (10). In particular, extraction mechanism (210) is incorporated into a portion of piercer drive (74). In other versions, extraction mechanism (210) may be incorporated into cutter drive (72), or both cutter drive (72) and piercer drive (74). In still other versions, extraction mechanism (210) may be entirely separate from drive assembly (70).

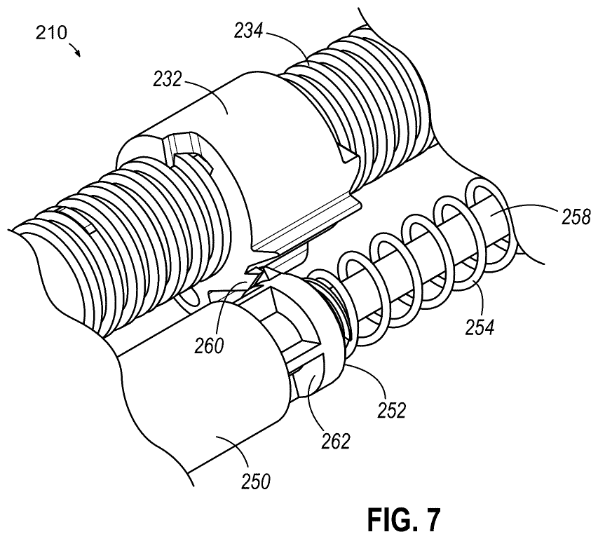

As can be seen in FIGS. 5 and 7, actuator (252) is configured to engage portions of piercer drive (74) such that piercer drive (74) may control one or more aspects of actuation of actuator (252). In particular, piercer drive (74) includes a lead screw (234) and a piercer carriage (232). Lead screw (234) and piercer carriage (232) are together configured to drive translation of piercer (22) via rotation of lead screw (234) and translation of piercer carriage (232) along the length of lead screw (234). Such translation of piercer carriage (232) may likewise be used to control one or more aspects of actuation of actuator (252) as will be described in greater detail below. In some versions, lead screw (234) and piercer carriage (232) may be configured in accordance with one or more of the teachings of US Pub. No. 2022/0249075, published on Aug. 11, 2022, the disclosure of which is incorporated by reference herein.

As best seen in FIG. 7, actuator (252) may be configured to releasably couple to piercer carriage (232). In particular, piercer carriage (232) may include a laterally extending latch (260) configured to releasably couple with a corresponding latch lip (262) of actuator (252). Through this, movement of actuator (252) may be selectively linked to movement of cutter carriage (232) and, by extension, piercer (22). In some versions, actuator (252) may include multiple latch lips (262) about perimeter of actuator (252).

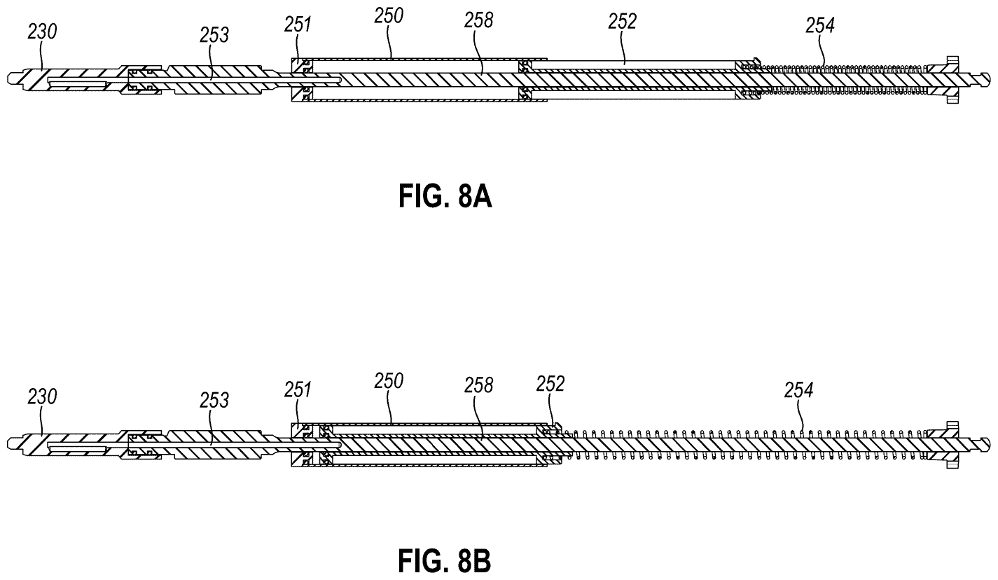

FIGS. 8A-8B depict a series of actuator (252) transitioning from the proximal cocked position to the distal released (also referred to as fired) position. FIG. 8A shows actuator (252) in the cocked position with actuator spring (254) in a compressed orientation such that fluid source (250) may be substantially filled or fully filled with a fluid or gas. Once actuator (252) begins to transition from the cocked position, as shown in FIG. 8A, to the released (also referred to as fired) position, as shown in FIG. 8B, the fluid or gas in fluid source (250) may begin to travel through lumen (253) of fluid conduit (258). A portion of lumen (253) may be positioned proximally of fluid source connector (251) such that lumen (253) is in fluid communication with fluid source (250). Fluid conduit (258) may include an opening into fluid source (250) that is just proximal to fluid source connector (251). In this configuration, actuator (252) may be capable of transitioning entirely distally into fluid source (250) such that there is no or a minimal amount of a fluid volume in fluid source (250), i.e. no or a minimal amount of dead space remaining in fluid source (250), and that any fluid or gas that was in fluid source (250) is forced into lumen (253) and out of nozzle (230). Should fluid source hold a compressible gas rather than a fluid, minimizing dead space can be advantageous to ensure maximum pressure in the gas throughout the actuator (252) stroke.

In order to transition actuator (252) into the cocked position shown in FIG. 8A, catch (260) of piercer carriage (232) may releasably couple with latch lip (262) while piercer carriage (232) is being retracted to retract piercer (220). Once piercer carriage (232) is coupled with actuator (252), lead screw (234) may rotate about an axis to continue translation of piercer carriage (232), which will likewise retract actuator (252). Actuator (252) may remain releasably coupled with piercer carriage (232) during this transition such that actuator (252) can also transition from a distal position to a proximal position which can thereby compress actuator spring (254) between actuator (252) and a fixed point such as housing (14) of biopsy device (10) or release gear (256) and apply a distally directed force on actuator (252).

While actuator (252) transitions from a distal to a proximal position, actuator (252) may be capable of producing a vacuum within fluid source (250) such that a gas or liquid is pulled into fluid source (250) to be subsequently expelled from nozzle (230) upon a release of actuator (252). In the present version, actuator (252) is configured to pull atmospheric air into the interior of fluid source (250) through nozzle (230). In other versions, a separate fluid reservoir may be coupled to fluid source (250) to permit any suitable alternative fluid to be pulled into fluid source (250) by actuator (252). Suitable alternative fluids may include, for example, saline, water, various oils, inert gasses, and/or etc.

Once actuator (252) is fully retracted to the proximal position, it may be desirable to decouple actuator (252) from piercer carriage (232) to thereby release actuator (252). Such decoupling may be completed in a variety of ways. For instance, in some versions, decoupling may be automatically driven by a differential between the travel distance of actuator (252) and the travel distance of piercer carriage (232). In such versions, piercer carriage (232) may continue translating distally after actuator (252) reaches a hard stop. One or more portions of latch (260) or latch lip (262) may be configured with a resilient, yet flexible portion to permit release of actuator (252) from piercer carriage (232) when a predetermined force is exceeded. In yet other versions, decoupling may be controlled by an external mechanism such as rotation of fluid conduit (258). For instance, in such versions, fluid conduit (258) may include one or more protrusions configured to rotate actuator (252). Similarly, one or more portions of latch (260) or latch lip (262) may include a cam feature configured to respond to rotation of actuator (252). Fluid conduit (258) may then be rotated by release gear (256) to rotate actuator (252), causing latch (260) and latch lip (262) to disengage. Still other suitable mechanisms may be used to facilitate decoupling as will be appreciated by those of ordinary skill in the art in view of the teachings herein.

Once actuator (252) is uncoupled with receiving portion (232) in the cocked position, actuator spring (254) may begin to expand and drive actuator (252) distally into fluid source (250). As actuator (252) is advanced into fluid source (250), any gas or fluid in fluid source (250) may be forced into lumen (253) of fluid conduit (258). The gas or fluid may travel through lumen (253) and travel into nozzle (230). Nozzle (230) may be a separate component to fluid conduit (258) and a junction between them may be sealed. Gas or fluid is then expelled from nozzle (230) in a direction towards notch (26) of piercer (22). The expelling of gas or fluid from nozzle (230) towards notch (26) can be enough to dislodge tissue from notch (26) and into a sample chamber. Nozzle (230) may be configured to direct fluid or gas directly into notch (26), directly onto tissue within notch (26), or may include a broad profile to clear notch (26) and portions surrounding notch (26) of any tissue.

Once actuator (252) has transitioned to the released (also referred to as fired) position and tissue has been cleared from notch (26), piercer (22) and piercer carriage (232) may be advanced distally in preparation of collecting another tissue sample. Once piercer carriage (232) has advanced distally, it may again releasably couple with actuator (252) to begin a new cycle. Once actuator (252) begins retraction to a proximal position, fluid source (250) may begin to refill based on a vacuum being applied from the removal of actuator (252). If fluid source (250) is filled with a gas, this gas may be taken from the surrounding environment and take a reversed path from the previous expulsion. In other words, as actuator (252) retracts proximally, ambient air may travel through nozzle (230), through lumen (253), and back into fluid source (250). If fluid source (250) is filled with a fluid, a separate fluid container (not shown) may be available to refill fluid source (250) as actuator (252) retracts out of fluid source (250).

### D. Example of Tissue Handler With Fluid Port

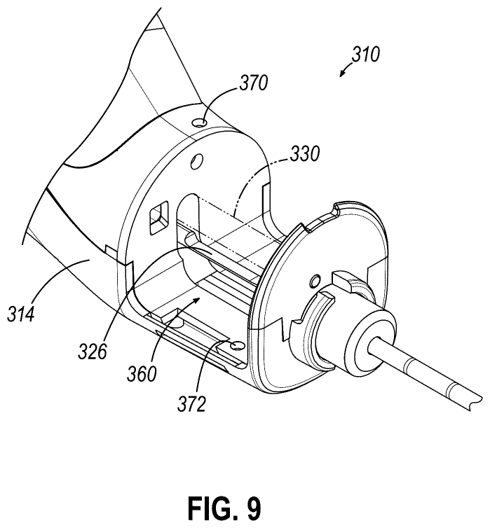

FIG. 9 shows an alternative extraction mechanism (310) described above in lieu of extraction mechanisms (110, 210). Extraction mechanism (310) includes a fluid port (370) that can be channeled to an outside of an upper portion of an outer housing (314). In other words, extraction mechanism (310) is configured for use with saline or other suitable fluids to manipulate a tissue sample. Fluid port (370) may be in fluid communication with an external gas or fluid supply (not shown) capable of supplying either gas or fluid to fluid port (370). Fluid port (370) may be in fluid communication with a nozzle (330). Nozzle (330) may include an opening directed towards a notch (326) of a piercer such that nozzle (330) is capable of expelling the fluid or gas towards notch (326) to thereby dislodge tissue from notch (326) and into a tissue collection feature (360). This example may be capable of sending multiple pulses of gas or fluid through fluid port (370) and nozzle (330) in an effort to dislodge a single piece of tissue. Expelling the fluid or gas through nozzle (326) may be controlled automatically by biopsy device (310) without operator input. Alternatively, expulsion may be controlled manually using a valve (not shown) within tissue handler (310) or by controlling pressure within the gas or fluid supply. Tissue collection feature (360) may include a valve or vent (372) to prevent the accumulation of fluid expelled from nozzle (330) or to prevent the build up of pressure within tissue collection feature (360). Valve or vent (372) may be removable coupled to an external collection device for collection of excess fluid passing through valve or vent (372). In some versions, fluid port (370) and nozzle (230) may be configured substantially similarly to fluid sources (150, 250), fluid conduits (158, 258), and/or fluid nozzles feature (130, 230) described above unless otherwise described herein.

Once tissue has been removed from notch (326), notch (326) may advance distally to capture an additional tissue sample. Upon proximal retraction of notch (326), nozzle (330) may be capable of removing additional tissue sample in the same manner as the first. Tissue handler (310) may be capable of taking an unlimited number of samples in this manner.

### II. Example Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A core needle biopsy device, comprising: a needle assembly, the needle assembly including a piercer and a hollow cutter, the piercer including a sharp distal tip and a notch proximal to the distal tip, the piercer being slidably disposed within the cutter to sever a tissue sample into the notch of the piercer; a drive assembly configured to selectively move the piercer and the cutter; and a tissue sample holder including a sample chamber, a fluid source, and a nozzle, the fluid source being configured to eject a liquid or gas from the nozzle and against a portion of the biopsy device to manipulate a severed tissue sample into the sample chamber.

### Example 2

The core needle biopsy device of example 1, the fluid source including a piston and a cylinder, the nozzle being secured to the fluid source such that advancement of the piston into the cylinder is configured to eject the liquid or gas from the nozzle to thereby manipulate the severed tissue sample into the sample chamber.

### Example 3

The core needle biopsy device of example 2, the fluid source further including a spring, the spring being configured to advance the piston into the cylinder.

### Example 4

The core needle biopsy device of any of examples 2 through 3, the fluid source further including a latch mechanism positioned on the piston or the cylinder, the latch mechanism being configured to translate with the piercer when the piercer is in a distal position and to move relative to the piercer when the piercer is in a proximal position.

### Example 5

The core needle biopsy device of example 4, the latch mechanism including a protrusion extending laterally from the cylinder.

### Example 6

The core needle biopsy device of any one or more of examples 2 through 5, the nozzle being in fluid communication with an interior of the cylinder.

### Example 7

The core needle biopsy device of any of examples 1 through 6, the fluid source being configured to eject a gas from the nozzle and against a portion of the biopsy device to manipulate the severed tissue sample into the sample chamber.

### Example 8

The core needle biopsy device of any of examples 1 through 7, the tissue sample holder further including a shaft extending distally from the nozzle and through the fluid source, a distal portion of the shaft having a lumen, the lumen being configured to place the nozzle in fluid communication with the fluid source.

### Example 9

The core needle biopsy device of example 8, the tissue sample holder further including a seal positioned between the nozzle and the shaft and configured to prevent leaking outside the nozzle and the shaft.

### Example 10

The core needle biopsy device of any of examples 1 through 9, the tissue sample holder further including an outer cover configured to removably couple to a housing of the biopsy device, the outer cover defining at least a portion of the sample chamber.

### Example 11

The core needle biopsy device of example 10, the outer cover including one or more walls defining the sample chamber.

### Example 12

The core needle biopsy device of examples 10 or 11, the outer cover including a piercer interface positioned proximate a portion of the piercer.

### Example 13

The core needle biopsy device of any of examples 10 through 12, the outer cover including one or more connectors configured to releasably secure the outer cover to the housing of the biopsy device.

### Example 14

The core needle biopsy device of any of examples 1 through 13, the tissue sample holder including a single nozzle.

### Example 15

The core needle biopsy device of any of examples 1 through 14, further comprising a body having a distal end, the needle assembly extending distally from the distal end of the body, the tissue sample holder being disposed on the distal end of the body.

### Example 16

A tissue sample holder for use with a core needle biopsy device, the core needle biopsy device including a piercer having a sample notch and a cutter movable relative to the sample notch to sever a tissue sample, the tissue sample holder comprising: a body defining a sample chamber, and a nozzle configured to manipulate a severed tissue sample from the sample notch of the piercer and into the sample chamber of the body.

### Example 17

The tissue sample holder of example 16, the body including an outer wall defining the sample chamber, the nozzle being configured to direct a liquid or gas within the outer wall of the body to move a severed tissue sample within the sample chamber.

### Example 18

The tissue sample holder of examples 16 or 17, the nozzle being in fluid communication with a gas or liquid line extending outside of the core needle biopsy device, the gas or liquid line being configured to pass gas or liquid through the nozzle to thereby manipulate a severed tissue sample from the sample notch of the piercer and into the sample chamber of the body.

### Example 19

The tissue sample holder of any of examples 16 through 18, the nozzle being configured to conduct a plurality of manipulations to the severed tissue sample from the sample notch of the piercer.

### Example 20

A method for collecting a tissue sample using a biopsy device, the method comprising: retracting a sample notch defined by a piercer proximally into a tissue sample holder; and ejecting a gas or liquid from a nozzle and towards the sample notch.

### Example 21

The method of example 20, where ejecting the gas or liquid from the nozzle and towards the sample notch manipulates a severed tissue sample from the sample notch of the piercer and into a sample chamber of a body.

### Example 22

The method of examples 20 or 21, the method further comprising advancing a piston into a cylinder to thereby eject the gas or liquid from the nozzle.

### Example 23

The method of example 22, further comprising retracting the piston from the cylinder to thereby fill the cylinder with the gas or liquid.

### Example 24

The method of example 23, the cylinder being filled with the gas or liquid by passing the gas or liquid through the nozzle.

### Example 25

A biopsy device, comprising: a body defined by a probe and a holster; a needle assembly extending distally from the probe, the needle assembly being configured to sever a tissue sample; and a tissue sample holder having a body defining sample chamber, a drive member, and a nozzle extending from a portion of the drive member, the drive member being configured to pressurize a liquid or gas and the nozzle being configured to eject the liquid or gas to thereby propel a tissue sample from the needle assembly and into the sample chamber.

### Example 26

The biopsy device of example 25, the drive member including a cylinder and piston, the piston being removably coupled to the needle assembly, the piston being configured to retract from the cylinder dependent of the needle assembly and to advance into the cylinder independent of the needle assembly.

### Example 27

The biopsy device of examples 25 or 26, the needle assembly including a cutter and a piercer, the piercer being coaxially disposed within the cutter, the piercer including a notch configured to receive the tissue sample.

### Example 28

The biopsy device of example 27, the nozzle being configured to align with the notch of the piercer.

### Example 29

A tissue sample holder for use with a core needle biopsy device, the core needle biopsy device including a piercer having a sample notch and a cutter movable relative to the sample notch to sever a tissue sample, the tissue sample holder comprising: a body defining a sample chamber, and a nozzle configured to manipulate a severed tissue sample from the sample notch of the piercer and into the sample chamber of the body.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

1. A core needle biopsy device, comprising:
(a) a needle assembly, the needle assembly including a piercer and a hollow cutter, the piercer including a sharp distal tip and a notch proximal to the distal tip, the piercer being slidably disposed within the cutter to sever a tissue sample into the notch of the piercer;
(b) a drive assembly configured to selectively move the piercer and the cutter; and
(c) a tissue sample holder including a sample chamber, a fluid source, and a nozzle, the fluid source being configured to eject a liquid or gas from the nozzle and against a portion of the biopsy device to manipulate a severed tissue sample into the sample chamber.

2. The core needle biopsy device of claim 1, the fluid source including a piston and a cylinder, the nozzle being secured to the fluid source such that advancement of the piston into the cylinder is configured to eject the liquid or gas from the nozzle to thereby manipulate the severed tissue sample into the sample chamber.

3. The core needle biopsy device of claim 2, the fluid source further including a spring, the spring being configured to advance the piston into the cylinder.

4. The core needle biopsy device of claim 2, the fluid source further including a latch mechanism positioned on the piston or the cylinder, the latch mechanism being configured to translate with the piercer when the piercer is in a distal position and to move relative to the piercer when the piercer is in a proximal position.

5. The core needle biopsy device of claim 4, the latch mechanism including a protrusion extending laterally from the cylinder.

6. The core needle biopsy device of claim 2, the nozzle being in fluid communication with an interior of the cylinder.

7. The core needle biopsy device of claim 1, the fluid source being configured to eject a gas from the nozzle and against a portion of the biopsy device to manipulate the severed tissue sample into the sample chamber.

8. The core needle biopsy device of claim 1, the tissue sample holder further including a shaft extending distally from the nozzle and through the fluid source, a distal portion of the shaft having a lumen, the lumen being configured to place the nozzle in fluid communication with the fluid source.

9. The core needle biopsy device of claim 8, the tissue sample holder further including a seal positioned between the nozzle and the shaft and configured to prevent leaking outside the nozzle and the shaft.

10. The core needle biopsy device of claim 1, the tissue sample holder further including an outer cover configured to removably couple to a housing of the biopsy device, the outer cover defining at least a portion of the sample chamber.

11. The core needle biopsy device of claim 10, the outer cover including one or more walls defining the sample chamber.

12. The core needle biopsy device of claim 10, the outer cover including a piercer interface positioned proximate a portion of the piercer.

13. The core needle biopsy device of claim 10, the outer cover including one or more connectors configured to releasably secure the outer cover to the housing of the biopsy device.

14. The core needle biopsy device of claim 1, the tissue sample holder including a single nozzle.

15. The core needle biopsy device of claim 1, further comprising a body having a distal end, the needle assembly extending distally from the distal end of the body, the tissue sample holder being disposed on the distal end of the body.

16. A tissue sample holder for use with a core needle biopsy device, the core needle biopsy device including a piercer having a sample notch and a cutter movable relative to the sample notch to sever a tissue sample, the tissue sample holder comprising:
a body defining a sample chamber, and a nozzle configured to manipulate a severed tissue sample from the sample notch of the piercer and into the sample chamber of the body.

17. The tissue sample holder of claim 16, the body including an outer wall defining the sample chamber, the nozzle being configured to direct a liquid or gas within the outer wall of the body to move a severed tissue sample within the sample chamber.

18. The tissue sample holder of claim 16, the nozzle being in fluid communication with a gas or liquid line extending outside of the core needle biopsy device, the gas or liquid line being configured to pass gas or liquid through the nozzle to thereby manipulate a severed tissue sample from the sample notch of the piercer and into the sample chamber of the body.

19. The tissue sample holder of claim 16, the nozzle being configured to conduct a plurality of manipulations to the severed tissue sample from the sample notch of the piercer.

20. A method for collecting a tissue sample using a biopsy device, the method comprising:
retracting a sample notch defined by a piercer proximally into a tissue sample holder; and
ejecting a gas or liquid from a nozzle and towards the sample notch.

21-29. (canceled)
